(19) United States
(12) Patent Application Publication
(10) Pub. No.: US 2008/0182781 A1
(43) Pub. Date: Jul. 31, 2008

(54) Process for the Preparation of Peptides

(76) Inventors: Divya Lal Saksena, Mumbai (IN); Shrikant Mishra, Mumbai (IN); Chandrakesan Muralidharan, Mumbai (IN); Milind Thakare, Mumbai (IN); Nilesh Patil, Mumbai (IN); Aruna Khare, Mumbai (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/592,118

(22) PCT Filed: Oct. 10, 2004
(86) PCT No.: PCT/IN04/00315

§ 371 Date: Aug. 15, 2007

(30) Foreign Application Priority Data
Jun. 14, 2004 (IN) 648/MUM/2004

(51) Int. Cl. A61K 38/12; C07K 7/64; C07K 7/06

(52) U.S. Cl. 514/10; 530/317; 530/330; 530/329

(57) ABSTRACT

The present invention relates to an improved process for the preparation of N⁶-(aminoiminomethyl)-N²-(3-mercapto-1-oxopropyl-L-lysylglycyl-L-α-aspartyl-L-tryptophyl-L-prolyl-L-cysteinamide, cyclic(1→6)-disulfide of formula (1), which involves assembling amino acid residues and a thioalkyl carboxylic acid with appropriate protecting groups on a solid phase resin, cleaving the peptide thus obtained from the resin with concomitant removal of side chain protecting groups except Acm protecting group of thiol moiety to obtain peptide amide of formula (3), converting lysine residue of peptide amide of formula (3) having protected thiol group to homoarginine residue by guanylation to obtain peptide of formula (4), preparing silver peptide of formula (5), followed by simultaneous deprotection, obtaining silver peptide of formula (5) and oxidation of silver peptide to obtain crude peptide amide comprising compound of formula (1) and finally subjecting to chromatographic purification. The described process is simple, easy, environment friendly and cost effective.

### TECHNICAL FIELD

The present invention relates to an improved process for the preparation of N⁶-(aminoiminomethyl)-N²-(3-mercapto-1-oxopropyl-L-lysylglycyl-L-α-aspartyl-L-tryptophyl-L-prolyl-L-cysteinamide, cyclic(1→6)-disulfide of formula (1) using solid phase Fmoc-chemistry.

### BACKGROUND AND PRIOR ART REFERENCES OF THE INVENTION

U.S. Pat. No. 5,318,899 describes N⁶-(aminoiminomethyl)-N²-(3-mercapto-1-oxopropyl-L-lysylglycyl-L-α-aspartyl-L-tryptophyl-L-prolyl-L-cysteinamide, cyclic (1→)-disulfide of the formula (1) as a therapeutic agent for the treatment of, and prevention of, platelet-associated ischemic disorders. It binds to the platelet receptor glycoprotein (GP) of human platelets and inhibits platelet aggregation. Platelet aggregation is mediated by GP complex on the surface of the platelet membrane. It exists on the surface of unstimulated platelets in an inactive form. When platelets are activated by adhesion and the physiological agonists, the GP also becomes activated such that it becomes a receptor for fibrinogen, von Willebrand Factor (vWF), and fibronectin. However, it is the binding of fibrinogen and/or vWF that is believed to be principally responsible for platelet aggregation and thrombus formation in vivo. This teaches that substances which specifically inhibit the binding of fibrinogen or vWF to GP, inhibit platelet aggregation and could be candidates for inhibiting thrombus formation in vivo (Eric J. Topol, Tatiana V. Byzova, Edward F. Plow and The Lancet; Vol 353; Jan. 16, 1999; pg 227-231). This article describes the compound having platelet aggregation inhibition activity but does not teach the method to synthesize the molecule.

Antagonists of platelet glycoprotein IIb/IIa have an approved role in reducing the extent of thrombotic complications leading to myocardial damage during percutaneous coronary interventions (PCI).

Compound of formula (1) is a disulphide looped cyclic heptapeptide containing six amino acids and one mercaptopropionyl(desamino cysteinyl) residue. The disulfide bridge is formed between the cysteine amide and the mercaptopropionyl moieties. It is known to be produced by solution-phase peptide synthesis and purified by preparative reverse phase liquid chromatography and lyophilized (www.fda.gov/cder/foi/label/1998/20718Ibl.pdf).

In terms of peptide synthesis methodology, two major synthetic techniques dominate current practice. These are synthesis in solution (homogeneous phase) and synthesis on solid phase (heterogeneous phase). But solution phase route is more cumbersome as compared to the solid phase route as after each coupling the peptide formed has to be isolated, whereas in the solid phase synthesis, the excess reagents and by-products are washed off by simple filtration. In both, the desired peptide compound is prepared by the step-wise addition of amino acid moieties to a building peptide chain.

U.S. Pat. Nos. 5,958,732 and 5,318,899 claim about recombinant techniques to synthesize peptides like N⁶-(aminoiminomethyl)-N²-(3-mercapto-1-oxopropyl-L-lysylglycyl-L-α-aspartyl-L-tryptophyl-L-prolyl-L-cysteinamide, cyclic(1→6)-disulfide of the formula (1). The peptide obtained by this recombinant process is modified by solution phase synthesis for conversion of lysine residue to homoarginine residue. These patent documents also claim solid phase synthesis using Boc chemistry and the subject matter of these patents is fundamentally different from the present invention.

As compared to Boc-chemistry, Fmoc-chemistry based synthesis utilizes a mild procedure and because of the base lability of Fmoc group, acid-labile side-chain protecting groups are employed providing orthogonal protection. The rationale for use of protecting groups is that the energy of breaking a bond of a protecting group is lower than any other group.

U.S. Pat. No. 5,686,566, U.S. Pat. No. 5,686,567, U.S. Pat. No. 5,686,569, U.S. Pat. No. 5,686,570 and U.S. Pat. No. 5,756,451 deal with different PAI's in their salt or other forms of the compound of formula (1) but do not teach the process for its preparation using Fmoc solid phase synthesis.

Likewise, U.S. Pat. No. 5,759,999, U.S. Pat. No. 5,786,333, U.S. Pat. No. 5,770,564, U.S. Pat. No. 5,807,825, U.S. Pat. No. 5,807,828, U.S. Pat. No. 5,843,897, U.S. Pat. No. 5,968,902, and U.S. Pat. No. 5,935,926 describe the method of treating platelet-associated disorders and the process for the preparation of peptide amide of formula (1) using boc chemistry.

U.S. Pat. No. 5,344,783 and U.S. Pat. No. 5,851,839 deal with methods for selecting and identifying Platelet Aggregation Inhibitors (PAI) and disclose boc chemistry for the preparation of peptide amide of formula (1).

U.S. Pat. No. 5,780,595 claims antibodies specific to PAI's and also discloses boc chemistry for the preparation of the peptide amide of formula (1).

The Fmoc route of synthesis of various other peptides is well-known in prior art and several documents are available for their preparation. However there is a definite need to develop a process for the preparation of compound of formula (1) which is economical, involves minimal steps and also eco-friendly.

As explained earlier, Fmoc-chemistry based synthesis utilises a mild procedure and because of the base lability of Fmoc group, acid-labile side-chain protecting groups are employed providing orthogonal protection. The protecting groups used in Fmoc chemistry are based on the tert-butyl moiety: tert-butyl ethers for Ser, Thr, tert-butyl esters for Asp, Glu and Boc for Lys, His. The trt and acm groups have been used for the protection of Cys. The guanidine group of Arg and Har is protected by Mtr, Pmc or Pbf. Most of the Fmoc-amino acids derivatives are commercially available. However, a problem exists in the art for the preparation of some amino acid analogs like peptides containing homoarginine as well as cyclic peptide compounds based on disulfide links, because separate operations are required before purifying the end product, which increases expense and may affect final product purity and yield. Fmoc-homoarginine residue if purchased commercially for use in the assembly of the chain becomes expensive. Alternatively in the peptide assembly, the Har unit is built by guanylation of the lysine residue at the α-NH₂, which has been demonstrated to obtain vasopressin analogues for the evaluation of its biological activity (Lindeberg et al, Int. J. Peptide Protein Res. 10, 1977, 240-244).

WO 03/093302 discloses the synthesis of the peptide of formula (1) using Fmoc-α-nitrogen protected Cα-carboxamide cysteine. It describes the attachment of the first amino acid, cysteine in the precipitated form to the solid support 4-methoxytrityl polystyrene resin through its thiol side chain, followed by removing the α-nitrogen protecting group and assembling the peptide on the said nitrogen. However, the process uses the solid support—4-methoxytrityl polystyrene resin which is not a common commercial embodiment and also the Fmoc-α-nitrogen protected Cα-carboxamide cysteine is not commercially available. This enables the process having increased number of steps and also expensive with respect to the process of the present invention. The cleavage conditions utilize ethanedithiol, which makes the process highly toxic and non-environment friendly requiring the use of expensive scrubbers. The use of Fmoc-homoarginine residue in the assembly of the chain is mentioned, which if purchased commercially, also makes the process very expensive. Overall, the process claimed in this document is different from the process claimed in the present invention. In addition the process of WO 03/093302 is associated with certain limitations, which has been overcome by providing suitable modifications in the process steps of the present invention.

Thus process of the present invention is an improved and efficient process over the one described in WO03/093302-A2 patent publication as herein mentioned below.
1 Does not involve the production of —SH peptide, which is susceptible to aerial oxidation leading to the formation of impurities, which hamper the purification of the final product and yield.
2 Precise selection of protecting groups for amino acids to build the peptide chain.
3 Activation of carboxylic function of the amino acid using appropriate activating reagent to prevent the racemization of amino acids.
4 Efficient process for obtaining disulfide loop in the peptide amide of formula (1) from silver-peptide salt intermediate without isolating —SH peptide.

A considerable number of known, naturally occurring small and medium-sized cyclic peptides as well as some of their synthetic derivatives and analogs possessing desirable pharmacological properties have been synthesized. However, wider medical use is often hampered due to complexity of their synthesis and purification. Therefore, improved methods for making these compounds in simple, lesser steps and at lesser cost are desirable and it is the need of the industry and mankind.

The purity and yield of the peptide are important aspects of any route of synthesis. Yield, represented by the relative content of the pharmacologically active compound in the final product, should be as high as possible. Purity is represented by the degree of presence of pharmacologically active impurities, which though present in trace amounts only, may disturb or even render useless the beneficial action of the peptide when used as a therapeutic agent. In a pharmacological context both aspects have to be considered. As a rule, purification becomes increasingly difficult with larger peptide molecules. In homogeneous (solution) phase synthesis (which is the current method of choice for industrial production of larger amounts of peptides) repeated purification required between individual steps provides a purer product but low yield. Thus, improvements in yield and purification techniques at the terminal stages of synthesis are needed. The present invention is an industrially feasible solid phase synthesis and is a novel process to yield a high purity product with enhanced yield.

Prior art describes the use of HOBt and DIC for activation of amino acids, which leads to the formation of the OBt ester. However, a major drawback in using this procedure is that the preparation of the OBt ester itself takes about 20 min and also the reaction has to be carried out at 0° C. The step-wise introduction of Nα-protected amino acids in SPPS normally involves in situ carboxyl group activation of the incoming amino acid or the use of pre-formed activated amino acid derivatives. In recent years, aminium and phosphonium based derivatives (HBTU, TBTU, Py Boc, and HATU) have become the preferred tools for in situ carboxyl activation. They have been shown to give superior results in terms of both coupling efficiency and suppression of enantiomerization. (Fmoc Solid Phase Peptide Synthesis by Chan W. C. and White P. D., Oxford University Press, 2000, p. 41-74) Use of HBTU provides high yield and high purity. It saves time in the activation step with no cooling required. Double coupling is also not required for Mpr(Acm)-OH.

Most of the Fmoc-amino acids derivatives are commercially available. However, a problem exists in the art for the preparation of some amino acid analogs like peptides containing homoarginine as well as cyclic peptide compounds based on disulfide links, because separate operations are required before purifying the end product, which increases expense and may affect final product purity and yield. Fmoc-homoarginine residue if purchased commercially for use in the assembly of the peptide chain becomes very expensive. Alternatively the peptide assembly can be built using lysine followed by guanylation of the lysine residue at the α-NH₂ (Lindeberg et al, Int. J. Peptide Protein Res. 10, 1977, 240-244).

Oxidative cyclization of protected or non-protected sulfhydryl groups with formation of disulfide structures is usually carried out as the final synthetic step, the reason being substantial thermal and chemical lability of the disulfide linkage. In few cases it is also carried out before cleavage of the peptide molecule from the solid support. The oxidation of open-chain peptides containing free and/or certain types of protected sulfhydryl groups with iodine in, e.g., methanol or acetic acid is further explained in the CRC Handbook of Neurohypophyseal Hormone Analogs, Vol. 1, Part 1; Jost, K., et al. Eds., CRC Press, Boca Raton, Fla. 1987, p. 31. Iodine, however, is not without drawbacks as a cyclization agent. For instance, tryptophan moieties present in peptide substrates are at risk of being iodinated, making the balance between full conversion of starting materials and minimizing side reactions a delicate one, which, in turn, impacts product purity. Tam (Tam J. P. et al., 1990, J. Am. Chem. Soc., Vol. 113, p. 6657) has demonstrated that the use of 20-50% solutions of DMSO in a variety of buffer systems greatly promotes disulfide bond formation in comparison with other methods such as aerial oxidation. DMSO is also found to greatly reduce and in some instances, suppress completely, the aggregation and precipitation of peptides that occurred using other oxidative procedures. Thus, the yield and purity of the disulfide looped peptide oxidized by DMSO is much higher as compared to other known methods. In the present invention this aspect has been rightfully tackled by not opting for Iodine route for oxidative cyclization. Also in the present invention the silver salt of peptide amide in place of peptide amide containing thiol group is subjected to oxidation without isolation of SH-peptide and eliminating the formation offside products during oxidation reaction. Thus the process steps of deprotection followed by oxidation of guanylated peptide amide adopted in the present invention yields crude peptide amide comprising compound of formula (1) of enhanced purity and yield. Finally purification of the crude peptide result in enhanced yield of the final pure peptide.

Another complicating factor in known routes of synthesis is the possibility of interaction between the desired cyclic disulfide and inorganic sulfur compounds used for reducing excess iodine at the end of the reaction, such as sodium dithionite or sodium thiosulfate. Such reducing sulfur-containing compounds may interact with the disulfide linkage, which is sensitive to nucleophilic attack in general. As the process of the present invention has avoided use of iodine, the resulting products have high purity and related impurities are undetectable.

The process is accomplished in a few easy and simple steps. The use of solid phase synthesis makes the process simpler and the use of Fmoc-chemistry eliminates the use of harsh chemicals like HF, thereby not affecting the product stability. The process eliminates purification of the intermediates, thereby increasing the yield and reducing the cost. Replacement of thiols as scavengers in step (b) and (e) makes the process more environment friendly and economical by not having to use scrubbers for thiols.

The choice of process often dictates the stability of the therapeutic peptide. There has been a long awaited requirement for obtaining peptide of formula (1) which will circumvent the limitations associated with the processes of prior art. Therefore, an industrial process of peptide synthesis containing tryptophan, disulfide loops, ε-NH₂ side chain, etc demands appropriate choice of protecting groups and reaction conditions to build up the peptide chain. This objective has been now successfully achieved by the Applicant developing a process described in entirety in the present application.

Glossary of Terms Used in the Specification
AA Amino Acid
Acm Acetamidomethyl
ACT Activator
ADP Adenosine diphosphate
AgOTf Silver trifluoromethane sulfonate
Arg Arginine
Asp Aspartic Acid
Boc/boc tert-butyloxycarbonyl
Cys Cysteine
DCM Dichloromethane
DEP Deprotection reagent
DMF Dimethyl Formamide
DMSO Dimethyl sulphoxide
DTT Dithiothreitol
EDT Ethane dithiol
Fmoc 9-fluorenylmethyloxycarbonyl
Glu Glutamic acid
Gly Glycine
HBTU 2-(1H-Benzotriazole-1-yl)-1,1,3,3-tetramethyluronium hexafluorophosphate
HF Hydrogen Fluoride
HIC Hydrophobic Interaction Chromatography
His Histidine
IEC Ion Exchange Chromatography
LC-MS Liquid Chromatography-Mass Spectroscopy
Lys Lysine
Mpr Mercaptopropionic Acid
Mtr 4-methoxy-2,3,6-trimethylbenzenesulfonyl
NMM N-methyl morpholine
Obut O-t-butyl
Pbf 2,2,4,6,7-pentamethyldihydrobenzofuran-5-sulfonyl
Pmc 2,2,5,7,8-pentamethylchroman-6-sulfonyl
PPP Platelet poor plasma
Pro Proline
PRP Platelet rich plasma
RP-HPLC Reverse Phase High Performance Liquid Chromatography.
RV Reaction Vessel
Ser Serine
SOLV Solvent
SP Synthetic Peptide
TEA Triethylamine
TFA Trifluoroacetic acid
Thr Threonine
TIS Triisopropylsilane
Trp Tryptophan
Trt Trityl

### OBJECTS OF THE INVENTION

The main object of the present invention is to provide an improved process to obtain N⁶-(aminoiminomethyl)-N²-(3-mercapto-1-oxopropyl-L-lysylglycyl-L-α-aspartyl-L-tryptophyl-L-prolyl-L-cysteinamide, cyclic(1→6)-disulfide of formula (1).

Another object of the present invention is to disclose a process for obtaining high yield and high purity of peptide amide of formula (1)

Yet another objective of the present invention is to disclose a process of solid phase synthesis of peptide amide of formula (1) by using Fmoc chemistry.

Still another object of the present invention is to disclose a process for the production of peptide of formula (1), having lesser number of steps as compared to solution phase synthesis.

Yet another object of the present invention is to design a process for the production of peptide amide of formula (1), which is devoid of limitations associated with prior art solid phase synthesis of compound of formula (1).

Still yet another object is to provide a process for preparing small and medium-size peptides containing a disulfide moiety having enhanced purity

Still yet another object of the present invention is to select appropriate protecting groups and reagents to minimize the formation of accompanying impurities in process steps, thereby enhancing the yield and reducing the cost.

### SUMMARY OF THE INVENTION

The present invention relates to an improved process for the preparation of N⁶-(aminoiminomethyl)-N²-(3-mercapto-1-oxopropyl-L-lysylglycyl-L-α-aspartyl-L-tryptophyl-L-prolyl-L-cysteinamide, cyclic(1→6)-disulfide of formula (1), which involves assembling amino acid residues and a thioalkyl carboxylic acid with an appropriate protecting groups on a solid phase resin, cleaving the peptide thus obtained from the resin with concomitant removal of side chain protecting groups except Acm protecting group of thiol moiety to obtain peptide amide of formula (3), converting lysine residue of peptide amide of formula (3) having protected thiol group to homoarginine residue by guanylation, followed by simultaneous deprotection, obtaining silver peptide of formula (5) and oxidation of silver peptide to obtain crude peptide amide of formula (1) and finally subjecting to chromatographic purification. The described process is simple, easy, environment friendly and cost effective.

### BRIEF DESCRIPTION OF FIGURES AND TABLE

FIG. 1: Analytical RP-HPLC elution profile of HBTU—crude peptide from resin (Column: PEP 300; C-18; 5μ; 150×3 mm; Flow rate: 0.5 ml/min; Injection vol: 20 μl; Solvent System: A: 0.1% TFA, B: 100% Acetonitrile).

FIG. 2: Analytical RP-HPLC elution profile of DIC—crude peptide from resin (Column: PEP 300; C-18; 5μ; 150×3 mm; Flow rate: 0.5 ml/min; Injection vol: 20 μl; Solvent System: A: 0.1% TFA, B: 100% Acetonitrile).

FIG. 3: Analytical RP-HPLC elution profile of crude guanylated peptide (Column: PEP 300; C-18; 5μ; 150×3 mm; Flow rate: 0.5 ml/min; Injection vol: 20 μl; Solvent System: A: 0.1% TFA, B: 100% Acetonitrile).

FIG. 4: Analytical RP-HPLC elution profile of SH peptide (Column: PEP 300; C-18; 5μ; 150×3 mm; Flow rate: 0.5 ml/min; Injection vol: 20 μl; Solvent System: A: 0.1% TFA, B: 100% Acetonitrile). Peak A—crude SH peptide.

FIG. 5: Analytical RP-HPLC elution profile of crude cyclic peptide (Column: PEP 300; C-18; 5μ; 150×3 mm; Flow rate: 0.5 ml/min; Injection vol: 20 μl; Solvent System: A: 0.1% TFA, B: 100% Acetonitrile); Peak A—crude cyclic peptide.

FIG. 6: Preparative RP-HPLC purification elution profile of crude cyclic peptide (Column: Phenomenex Luna; C-18(2); 10μ; 250×50 mm; Flow rate: 50 ml/min; Solvent System: A: 0.1% TFA, B: 100% Methanol).

FIG. 7: Analytical RP-HPLC elution profile of purified cyclic peptide (Column: PEP 300; C-18; 5μ; 150×3 mm; Flow rate: 0.5 ml/min; Solvent System: A: 0.1% TFA, B: 100% Acetonitrile); Peak A—purified peptide.

FIG. 8: MS Analysis of the pure peptide showing the mass to be 832 and impurity to be 903

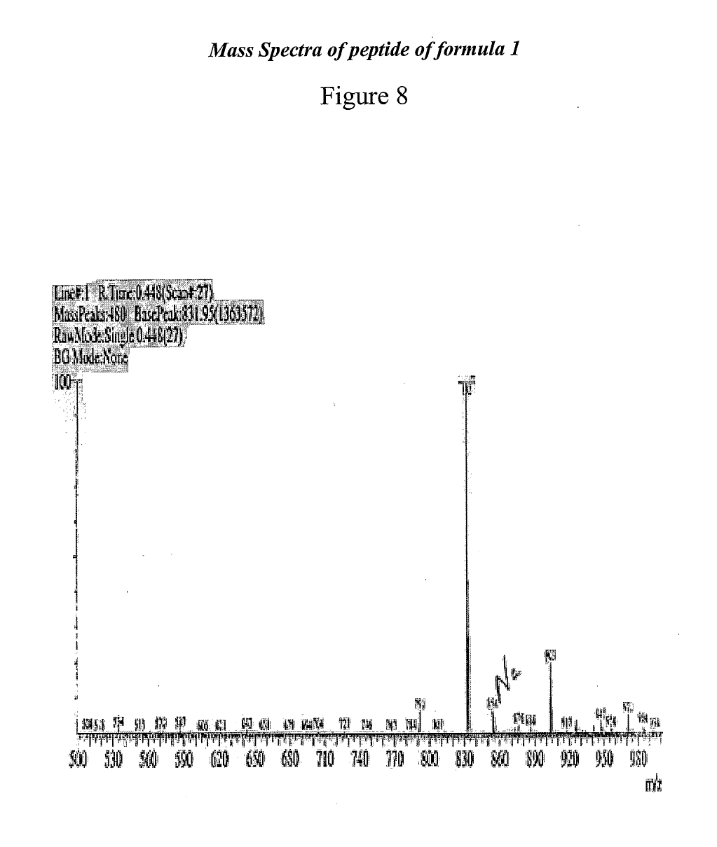

Table 1: Inhibition of ADP induced aggregation by synthesized peptide of formula (1).

### DETAILED DESCRIPTION OF THE INVENTION

In accordance, the present invention provides a. process for the preparation of a peptide N⁶-(aminoiminomethyl)-N²-(3-mercapto-1-oxopropyl-L-lysylglycyl-L-α-aspartyl-L-tryptophyl-L-prolyl-L-cysteinamide, cyclic(1→6)-disulfide of formula (1) on a solid phase, the said process comprising steps of, 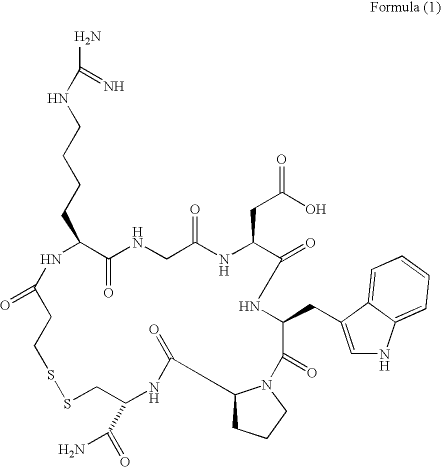
a) assembling a peptide chain comprising of six amino acids and a thioalkyl carboxylic acid in a required sequence on a solid support resin by coupling to directly join one another by peptide bonds to obtain peptide of formula (2);
   (Acm)Mpr-Lys(Boc)-Gly-Asp(Obut)-Trp-Pro-Cys(Acm)-Resin Formula (2)
b) capping the free amino groups of step (a) after each coupling with acetic anhydride;
c) cleaving and deprotecting, all groups except acm group in the peptide of step (b) from the solid support resin to obtain peptide-amide of formula (3);
   (Acm)Mpr-Lys-Gly-Asp-Trp-Pro-Cys(Acm)-CONH₂ Formula (3);
d) guanylating the peptide of step (c) at ε-lysine-NH₂ in an organic solvent followed by precipitating with an another solvent to obtain peptide-amide of formula (4);
   (Acm)Mpr-Homoarg-Gly-Asp-Trp-Pro-Cys(Acm)-CONH₂ Formula (4)
e) treating the peptide amide of Formula (4) of step (d) with a heavy metal salt in an appropriate solvent, followed by precipitating using an organic solvent to obtain the heavy metal-peptide salt of formula (5); 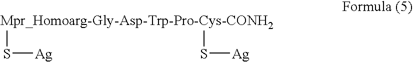
f) treating the heavy metal-peptide salt of step (e) with an appropriate nucleophilic reagent to obtain the crude peptide amide of formula (1); and
g) purifying the crude peptide amide of step (f) by chromatographic techniques.

An embodiment of the present invention involves reaction of amino and carboxylic equivalent of compounds to form the said peptide bond.

Another embodiment of the present invention provides C-terminal of the protected first amino acid bound to a solid phase resin through a linker to obtain a solid phase bound amino acid.

Yet another embodiment of the present invention uses solid support has any amide resin, preferably Rink Amide Resin.

Still another embodiment of the present invention uses first protected amino acid as thiol protected Fmoc cysteine.

Yet another embodiment of the present invention uses HBTU as the coupling agent.

Still yet another embodiment of the present invention provides a cleavage of the resin with the linker leading to release of assembled peptide amide.

Yet another embodiment of the present invention provides peptide amide compound of formula (1) obtained by linking each of terminal functionality, which is an amino or carboxylic acid group or derivatives thereof.

Still another embodiment of the present invention uses amino acids selected from the group consisting of Cys, Pro, Trp, Asp, Lys, Gly, Arg, Har, Leu, Glu.

An embodiment of the present invention uses thioalkyl carboxylic acid 2-thiopropionic acid.

Another embodiment of the present invention provides the use of protecting groups for amino function of an amino acid as Fmoc or Boc.

Yet another embodiment of the present invention provides the use of carboxyl function as unprotected or protected O-tBu ester.

Still another embodiment of the present invention uses the protecting group for thiol-function as Acm group.

Still yet another embodiment of the present invention provides cleavage of the peptide from solid support resin using the reagents TFA, TIS, EDT, DCM, Phenol and water in a defined ratio, preferably TFA(85-98%):TIS(0-5%):H₂O(0-5%):EDT(0-5%):Phenol(0-5%), more preferably TFA(94.5-95.5%):TIS(0-2.5%):H₂O(0-3%) EDT(0-2.5%).

Another embodiment of the present invention utilizes an organic solvent for guanylation selected from a group consisting of DMF, ethanol and methanol.

Yet another embodiment of the present invention the guanylation of peptide is performed preferably by using the solvent DMF.

Still another embodiment of the present invention the precipitation of the peptide amide of formula (4) is performed using a solvent selected from the group consisting of acetone, acetonitrile, methanol, ethers, pentane, hexane and mixture thereof.

Still yet another embodiment of the present invention the precipitation is preferably performed using acetonitrile.

Another embodiment of the present invention) the purification of the peptide of formula (4) can be achieved by RP-HPLC.

Yet another embodiment of the present invention the peptide amide of formula (1) obtained has purity more than 99%.

Still yet another embodiment of the present invention the preparation of the peptide of formula (1) by solid phase synthesis is carried out using Fmoc chemistry.

Further embodiment of the present invention uses heavy metal salt for removal of acm selected from thallium trifluoromethane sulphonate, mercuric acetate or silver trifluoromethane sulphonate, preferably silver trifluoromethane sulphonate.

Another embodiment of the present invention the heavy metal peptide salt is obtained by preferably treating peptide of formula (4) with silver trifluoromethane sulphonate in TFA.

Yet another embodiment of the present invention the precipitation of the heavy metal-peptide salt of Formula (5) is preferably carried out using an etheral solvent and more preferably disopropyl ether.

Still another embodiment of the present invention the heavy metal-peptide salt may be treated with HCl and DMSO to simultaneously remove the heavy metal and to oxidize the resulting peptide to yield crude peptide amide of formula (1).

Still yet another embodiment of the present invention the crude peptide amide of formula (1) may be purified by RP-HPLC.

Another embodiment of the present invention the purification of crude peptide amide of formula (1) is preferentially performed by RP-HPLC using C-4, C-8 or C-18 silica or polymer reverse phase columns using methanol and/or acetonitrile in combination with aqueous TFA(0-0.5%) as mobile phase

Still another embodiment of the present invention uses methanol (AR grade) for purification of crude peptide enabling the process inexpensive.

Yet another embodiment of the present invention provides process for preparation of an intermediate peptide of formula (2) as given under:
(Acm)Mpr-Lys(Boc)-Gly-Asp(Obut)-Trp-Pro-Cys(Acm)-Resin Formula (2)

Still another embodiment of the present invention provides process for preparation of an intermediate peptide of amide formula (3) as given under:
(Acm)Mpr-Lys-Gly-Asp-Trp-Pro-Cys(Acm)-CONH₂ Formula (3)

Still yet another embodiment of the present invention provides process for, preparation of a peptide amide of formula (4) as given below:
(Acm)Mpr-Homoarg-Gly-Asp-Trp-Pro-Cys(Acm)-CONH₂ Formula (4)

Yet another embodiment of the present invention provides process for preparation of an intermediate peptide amide silver salt of formula (5) as given under: 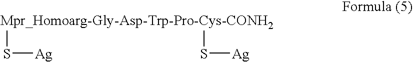
The following examples are illustrative of the present invention and not to be construed to limit the scope of the invention.

### EXAMPLES

### Example (1)

### Chemical Synthesis of Linear Peptide

(Acm)Mpr-Lys(Boc)-Gly-Asp(OBut)-Trp-Pro-Cys(Acm)-Resin Formula (2)
General Procedure:

The assembly of the peptide chain is carried out in the following manner. The resin is transferred to the RV of the peptide synthesizer [CS936, CS BIO, Calif. Peptide Synthesizer] and the linear peptide is assembled on it using 1.5-4.0 times mole excess amino acid derivatives, on the peptide synthesizer. The first amino acid, Fmoc-Cys (C), is coupled to the resin by deprotecting the Fmoc-group on the resin, followed by activating the Fmoc-Cys(C) by HBTU in the presence of NMM. For coupling of the next amino acid, Proline, the α-nitrogen of the first amino acid i.e. Fmoc-Cys(C), is deprotected followed by activating the Fmoc-Pro by HBTU in the presence of NMM. This process is repeated with all the amino acids till the entire linear peptide chain is assembled on the solid support. The Mpr is assembled at the end. Each coupling is carried out for a time range of 45-90 min. The coupling steps are followed by capping with acetic anhydride for 30-60 min. After the coupling are complete, the resin is washed with organic solvent/s which may be selected from the range of DMF, N-methylpyrrolidone or DCM, preferably DMF followed by DCM, and then dried under vacuum. The linear peptide of formula (2) is obtained.

The peptide was synthesized as peptide amide by solid phase peptide synthesis technology on rink amide resin using Fmoc chemistry.

| | |
|---|---|
| Instrument | CS936, CS BIO, Calif. Peptide synthesizer |
| Resin | Rink amide resin (0.65 mm/g) |
| Activator | HBTU/0.4M NMM |
| Solvent | Dimethyl Formamide |
| Deprotection | 20% Piperidine |

The resin (15.38 g-rink amide, 10 mmole) was transferred to the RV of the CS936 and swollen in DMF.
(i) Synthesis of Fmoc Cys(Acm)-resin by coupling of Fmoc-Cys(Acm)/HBTU to the resin. The pre-swollen resin (10 mmole) was washed twice with DMF followed by removal of Fmoc by treatment with 20% piperidine twice. The resin was washed 6 times with DMF. Fmoc Cys(Acm) (20 mmoles) and HBTU (equimole to amino acid) were dissolved in 0.4M NMM and added to the resin. Coupling was carried out for 60 min under optimized stirring. The resin was washed once again with DMF thrice. After the coupling, the free amino groups were capped by acetic anhydride (2.5M) for 45 min followed by washing with DMF three times. This HBTU process is a one-step process wherein ester is not isolated.

The synthesis cycle was programmed as follows:

| Step | Reagent | Time | Repeat | Activity |
|---|---|---|---|---|
| 1 | SOLV | 10 min | ×3 | WASHES RESIN |
| 2 | DEP | 5 min | ×2 | DEP N-TERMINUS |
| 3 | SOLV | 30 sec | ×6 | WASHES RESIN |
| 4 | ACT | 30 sec | ×1 | DISSOLVES Fmoc-Cys (Acm)/ |
| | | | | HBTU |
| 5 | AA | 45 min | ×1 | Fmoc-Cys (Acm) COUPLING |
| 6 | SOLV | 30 sec | ×3 | WASHES RESIN |

(ii) Synthesis of Fmoc-Pro-Cys(Acm)-resin by coupling Fmoc-Pro/HBTU to Fmoc-Cys(Acm)-resin. The reaction was carried out as in step 1. The synthesis cycle was programmed as follows:

| Step | Reagent | Time | Repeat | Activity |
|---|---|---|---|---|
| 1 | SOLV | 30 sec | ×3 | WASHES RESIN |
| 2 | DEP | 5 min | ×2 | DEP N-TERMINUS |
| 3 | SOLV | 30 sec | ×6 | WASHES RESIN |
| 4 | ACT | 30 sec | ×1 | DISSOLVES Fmoc-Pro/HBTU |
| 5 | AA | 45 min | ×1 | COUPLING Fmoc-Pro |
| 6 | SOLV | 30 sec | ×3 | WASHES RESIN |

(iii) Synthesis of Fmoc-Trp-Pro-Cys(Acm)-resin by coupling Fmoc-Trp/HBTU to Fmoc-Pro-Cys(Acm)-resin. The reaction was carried out as in step 1. The synthesis cycle was programmed as follows:

| Step | Reagent | Time | Repeat | Activity |
|---|---|---|---|---|
| 1 | SOLV | 30 sec | ×3 | WASHES RESIN |
| 2 | DEP | 5 min | ×2 | DEP N-TERMINUS |
| 3 | SOLV | 30 sec | ×6 | WASHES RESIN |
| 4 | ACT | 30 sec | ×1 | DISSOLVES Fmoc-Trp/HBTU |
| 5 | AA | 45 min | ×1 | COUPLING Fmoc-Trp |
| 6 | SOLV | 30 sec | ×3 | WASHES RESIN |

(iv) Synthesis of Fmoc-Asp(Obut)-Trp-Pro-Cys(Acm)-resin by coupling Fmoc-Asp (Obut)/HBTU to Fmoc-Trp-Pro-Cys(Acm)-resin. The reaction was carried out as in step 1. The synthesis cycle was programmed as follows:

| Step | Reagent | Time | Repeat | Activity |
|---|---|---|---|---|
| 1 | SOLV | 30 sec | ×3 | WASHES RESIN |
| 2 | DEP | 5 min | ×2 | DEP N-TERMINUS |
| 3 | SOLV | 30 sec | ×6 | WASHES RESIN |
| 4 | ACT | 30 sec | ×1 | DISSOLVES Fmoc-Asp(Obut)/ |
| | | | | HBTU |
| 5 | AA | 45 min | ×1 | COUPLING Fmoc-Asp(Obut) |
| 6 | SOLV | 30 sec | ×3 | WASHES RESIN |

(v) Synthesis of Fmoc-Gly-Asp (Obut)-Trp-Pro-Cys(Acm)-resin by coupling Fmoc-Gly/HBTU to Fmoc-Asp(Obut)-Trp-Pro-Cys(Acm)-resin. The reaction was carried out as in step 1. The synthesis cycle was programmed as follows:

| Step | Reagent | Time | Repeat | Activity |
|---|---|---|---|---|
| 1 | SOLV | 30 sec | ×3 | WASHES RESIN |
| 2 | DEP | 5 min | ×2 | DEP N-TERMINUS |
| 3 | SOLV | 30 sec | ×6 | WASHES RESIN |
| 4 | ACT | 30 sec | ×1 | DISSOLVES Fmoc-Gly/HBTU |
| 5 | AA | 45 min | ×1 | COUPLING Fmoc-Gly |
| 6 | SOLV | 30 sec | ×3 | WASHES RESIN |

(vi) Synthesis of Fmoc-Lys(Boc)-Gly-Asp(Obut)-Trp-Pro-Cys(Acm)-resin by coupling Fmoc-Lys(Boc)/HBTU to Fmoc-Gly-Asp(Obut)-Trp-Pro-Cys(Acm)-resin. The reaction was carried out as in step 1. The synthesis cycle was programmed as follows:

| Step | Reagent | Time | Repeat | Activity |
|---|---|---|---|---|
| 1 | SOLV | 30 sec | ×3 | WASHES RESIN |
| 2 | DEP | 5 min | ×2 | DEP N-TERMINUS |
| 3 | SOLV | 30 sec | ×6 | WASHES RESIN |
| 4 | ACT | 30 sec | ×1 | DISSOLVES Fmoc-Lys(Boc)/HBTU |
| 5 | AA | 45 min | ×1 | COUPLING Fmoc-Lys(Boc) |
| 6 | SOLV | 30 sec | ×3 | WASHES RESIN |

(vii) Synthesis of Mpr(Acm)-Lys(Boc)-Gly-Asp(Obut)-Trp-Pro-Cys(Acm)-resin by coupling Mpr(Acm)/HBTU to Fmoc-Lys(Boc)-Gly-Asp(Obut)-Trp-Pro-Cys(Acm)-resin. The reaction was carried out as in step 1. The synthesis cycle was programmed as follows:

| Step | Reagent | Time | Repeat | Activity |
|---|---|---|---|---|
| 1 | SOLV | 30 sec | ×3 | WASHES RESIN |
| 2 | DEP | 5 min | ×2 | DEP N-TERMINUS |
| 3 | SOLV | 30 sec | ×6 | WASHES RESIN |
| 4 | ACT | 30 sec | ×1 | DISSOLVES Mpr(Acm)/HBTU |
| 5 | AA | 45 min | ×1 | COUPLING Mpr(Acm) |
| 6 | SOLV | 30 sec | ×3 | WASHES RESIN |

### Example (2)

### Chemical Synthesis of Linear Peptide

(Acm)Mpr-Lys(Boc)-Gly-Asp(OBut)-Trp-Pro-Cys(Acm)-Resin Formula (2)
General Procedure:

The assembly of the peptide chain is carried out in the following manner. The resin is transferred to the RV of the peptide synthesizer [PS3, Protein Technologies, Peptide Synthesizer] and the linear peptide is assembled on it using 1.5-4.0 times mole excess amino acid derivatives, on the peptide synthesizer. The first amino acid, Fmoc-Cys (C), is coupled to the resin by deprotecting the Fmoc-group on the resin, followed by activation of Fmoc-Cys(C). Fmoc-Cys(C) (1.3 mmole) and HOBt (2.6 mmole) were dissolved in DMF (5.0 ml) and cooled to less than 10° C. in an ice bath. DIC (1.74 mmole) was added to the reaction mixture as a single aliquot. The mixture was then agitated for 6 minutes before being charged to the damp resin in the reaction vessel. The coupling reaction takes place for 60 mins.

For coupling of the next amino acid, Proline, the α-nitrogen of the first amino acid i.e. Fmoc-Cys(C), is deprotected. This is followed by activation of Fmoc-Pro by DIC/HOBt in cold conditions as described above and then transfer of this mixture to the reaction vessel. This process is repeated with all the amino acids till the entire linear peptide chain is assembled on the solid support. The Mpr is assembled at the end. Each coupling is carried out for a time range of 45-90 min. Coupling of Mpr is repeated. The coupling steps are followed by capping with acetic anhydride for 30-60 min. After the coupling are complete, the resin is washed with organic solvent/s which may be selected from the range of DMF, N-methylpyrrolidone or DCM, preferably DMF followed by DCM, and then dried under vacuum. The linear peptide of formula (2) is obtained.

The peptide was synthesized as peptide amide by solid phase peptide synthesis technology on rink amide resin using Fmoc chemistry.

| | |
|---|---|
| Instrument | PS3, Protein Technologies, Peptide synthesizer |
| Resin | Rink amide resin (0.65 mm/g) |
| Activator | DIC/HOBT |
| Solvent | Dimethyl Formamide |
| Deprotection | 20% Piperidine |

The resin (1 g-rink amide, 0.65 mmole) was transferred to the RV of the PS3 and swollen in DMF.

Synthesis of Fmoc Cys(Acm)-resin by coupling of activated Fmoc-Cys(Acm) to the resin. The pre-swollen resin (0.65 mmole) was washed twice with DMF followed by removal of Fmoc by treatment with 20% piperidine twice. The resin was washed 6 times with DMF. Fmoc Cys(Acm) (1.3 mmoles) and HOBt (2.6 mmole) were dissolved in DMF (5.0 ml) and cooled to less than 10° C. in an ice bath. DIC (1.74 mmole) was added to the reaction mixture as a single aliquot. The mixture was then agitated for 6 minutes before being charged to the damp resin. Coupling was carried out for 60 min under optimized stirring. The resin was washed once again with DMF thrice. After the coupling, the free amino groups were capped by acetic anhydride (2.5M) for 45 min followed by washing with DMF three times. This DIC/HOBt process is a manual and multistep process.

The synthesis cycle was programmed as follows:

| Step | Reagent | Time | Repeat | Activity |
|---|---|---|---|---|
| 1 | SOLV | 10 min | ×3 | WASHES RESIN |
| 2 | DEP | 5 min | ×2 | DEP N-TERMINUS |
| 3 | SOLV | 30 sec | ×6 | WASHES RESIN |
| 4 | Manual addition of activated Fmoc amino acid. | | | |
| 5 | AA | 45 min | ×1 | Fmoc-Cys (Acm) COUPLING |
| 6 | SOLV | 30 sec | ×3 | WASHES RESIN |

(ii) Synthesis of Fmoc-Pro-Cys(Acm)-resin by coupling activated Fmoc-Pro to Fmoc-Cys(Acm)-resin. The reaction was carried out as in step 1. The synthesis cycle was programmed as follows:

| Step | Reagent | Time | Repeat | Activity |
|---|---|---|---|---|
| 1 | SOLV | 30 sec | X3 | WASHES RESIN |
| 2 | DEP | 5 min | X2 | DEP N-TERMINUS |
| 3 | SOLV | 30 sec | X6 | WASHES RESIN |

| | |
|---|---|
| 4 | Manual addition of activated Fmoc amino acid. |

| | | | | |
|---|---|---|---|---|
| 5 | AA | 45 min | X1 | COUPLING Fmoc-Pro |
| 6 | SOLV | 30 sec | X3 | WASHES RESIN |

(iii) Synthesis of Fmoc-Trp-Pro-Cys(Acm)-resin by coupling activated Fmoc-Trp to Fmoc-Pro-Cys(Acm)-resin. The reaction was carried out as in step 1. The synthesis cycle was programmed as follows:

| Step | Reagent | Time | Repeat | Activity |
|---|---|---|---|---|
| 1 | SOLV | 30 sec | X3 | WASHES RESIN |
| 2 | DEP | 5 min | X2 | DEP N-TERMINUS |
| 3 | SOLV | 30 sec | X6 | WASHES RESIN |

| | |
|---|---|
| 4 | Manual addition of activated Fmoc amino acid. |

| | | | | |
|---|---|---|---|---|
| 5 | AA | 45 min | X1 | COUPLING Fmoc-Trp |
| 6 | SOLV | 30 sec | X3 | WASHES RESIN |

(iv) Synthesis of Fmoc-Asp(Obut)-Trp-Pro-Cys(Acm)-resin by coupling Fmoc-Asp to Fmoc-Trp-Pro-Cys(Acm)-resin. The reaction was carried out as in step 1. The synthesis cycle was programmed as follows:

| Step | Reagent | Time | Repeat | Activity |
|---|---|---|---|---|
| 1 | SOLV | 30 sec | X3 | WASHES RESIN |
| 2 | DEP | 5 min | X2 | DEP N-TERMINUS |
| 3 | SOLV | 30 sec | X6 | WASHES RESIN |
| 4 | Manual addition of activated Fmoc amino acid. | | | |
| 5 | AA | 45 min | X1 | COUPLING Fmoc-Asp(Obut) |
| 6 | SOLV | 30 sec | X3 | WASHES RESIN |

(v) Synthesis of Fmoc-Gly-Asp (Obut)-Trp-Pro-Cys(Acm)-resin by coupling Fmoc-Gly to Fmoc-Asp(Obut)-Trp-Pro-Cys(Acm)-resin. The reaction was carried out as in step 1. The synthesis cycle was programmed as follows:

| Step | Reagent | Time | Repeat | Activity |
|---|---|---|---|---|
| 1 | SOLV | 30 sec | X3 | WASHES RESIN |
| 2 | DEP | 5 min | X2 | DEP N-TERMINUS |
| 3 | SOLV | 30 sec | X6 | WASHES RESIN |
| 4 | Manual addition of activated Fmoc amino acid. | | | |
| 5 | AA | 45 min | X1 | COUPLING Fmoc-Gly |
| 6 | SOLV | 30 sec | X3 | WASHES RESIN |

(vi) Synthesis of Fmoc-Lys(Boc)-Gly-Asp(Obut)-Trp-Pro-Cys(Acm)-resin by coupling Fmoc-Lys(Boc) to Fmoc-Gly-Asp(Obut)-Trp-Pro-Cys(Acm)-resin. The reaction was carried out as in step 1. The synthesis cycle was programmed as follows:

| Step | Reagent | Time | Repeat | Activity |
|---|---|---|---|---|
| 1 | SOLV | 30 sec | X3 | WASHES RESIN |
| 2 | DEP | 5 min | X2 | DEP N-TERMINUS |
| 3 | SOLV | 30 sec | X6 | WASHES RESIN |
| 4 | Manual addition of activated Fmoc amino acid. | | | |
| 5 | AA | 45 min | X1 | COUPLING Fmoc-Lys(Boc) |
| 6 | SOLV | 30 sec | X3 | WASHES RESIN |

(vii) Synthesis of Mpr(Acm)-Lys(Boc)-Gly-Asp(Obut)-Trp-Pro-Cys(Acm)-resin by coupling Mpr(Acm) to Fmoc-Lys(Boc)-Gly-Asp(Obut)-Trp-Pro-Cys(Acm)-resin. The reaction was carried out as in step 1. The synthesis cycle was programmed as follows:

| Step | Reagent | Time | Repeat | Activity |
|---|---|---|---|---|
| 1 | SOLV | 30 sec | X3 | WASHES RESIN |
| 2 | DEP | 5 min | X2 | DEP N-TERMINUS |
| 3 | SOLV | 30 sec | X6 | WASHES RESIN |
| 4 | Manual addition of activated Fmoc amino acid. | | | |
| 5 | AA | 45 min | X1 | COUPLING Mpr(Acm) |
| 6 | SOLV | 30 sec | X3 | WASHES RESIN |

In the synthesis coupling of Mpr(Acm) had to be carried out twice to complete the coupling reaction.

### Example (3)

### Cleavage of the Peptide from the Resin to Yield Peptide Amide

(Acm)Mpr-Lys-Gly-Asp-Trp-Pro-Cys(Acm)-CONH₂ (Formula (3))

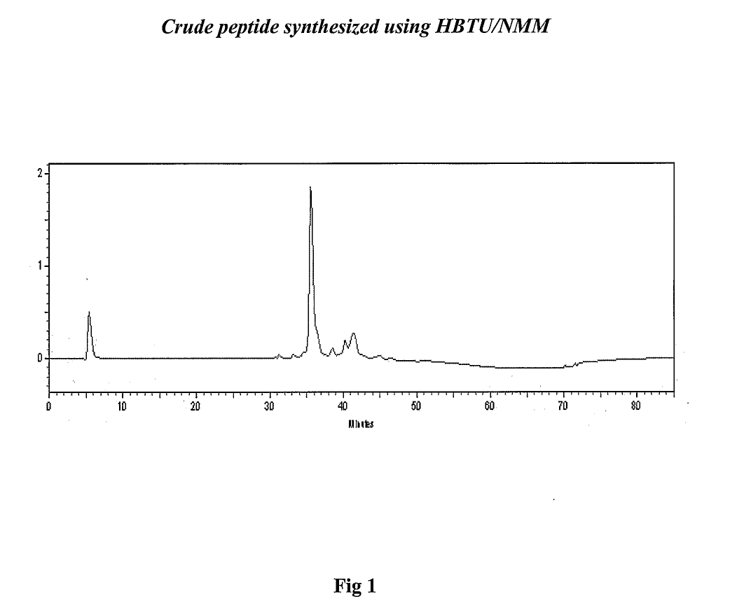

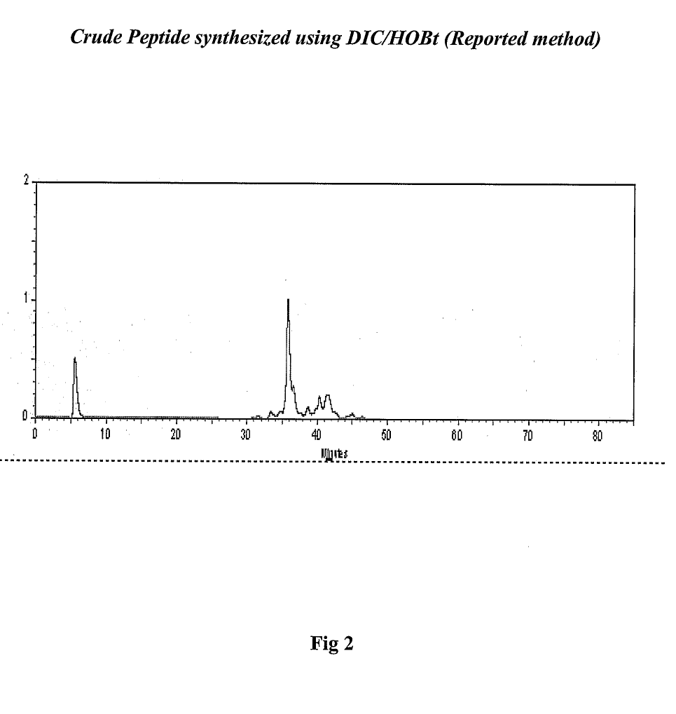

The assembled peptide resin (from Example 1 or 2) is treated with 500 ml of cleavage cocktail consisting of TFA (95%):TIS(2.5%):H₂O(2.5%):EDT(0%): Phenol (0%) for 2 hrs at R.T in CS936. The reaction mixture is filtered through RV, and TFA was evaporated on Rotavap. Precipitation of the peptide was carried out at −20° C. by addition of 300 ml of cold diisopropyl ether with constant stirring. The crude peptide precipitate in the solvent is let to stand at −20° C. for 10 hrs. The peptide was isolated by filtering through Whatman paper no. 5, followed by cold solvent wash (100 ml×3) to remove the scavengers used in the cleavage cocktail. The crude peptide precipitate is dried under vacuum over P₂O₅, and characterized by RP-HPLC (FIGS. 1 and 2).

| Example 1 | Example 2 |
|---|---|
| Yield: 58.73 | Yield: 48.73 |
| % purity of peptide: 90% | % purity of peptide: 79.68% |

### Example (4)

### Guanylation of Crude Peptide to Yield

(Acm)Mpr-Homoarg-Gly-Asp-Trp-Pro-Cys(Acm)-CONH₂ (formula (4))

The peptide (1 g, 1.157 mmole) is dissolved in 15 ml of DMF, the pH adjusted to 9.0 with TEA. The reagent 3,5-dimethylpyrazole-1-carboxamidine nitrate (931.5 mg) in DMF (15 ml) is added to the peptide. The reaction mixture is stirred at 30° C. for 4 days with multiple additions of one time excess of reagent 3,5-dimethylpyrazole-1-carboxamidine nitrate.

The peptide is precipitated from the reaction mixture by the addition of 280 ml of acetonitrile (pH adjusted to 8.0 with TEA). The mix is further kept at −20° C. for 10 hrs. It is filtered through Whatman no. 5 filter paper and washed with acetonitrile (pH 8.0) 3 times, followed by plain acetonitrile to neutralize the pH. The precipitate is dried under high vacuum overnight. Yield: 85%. The peptide was characterized by RP-HPLC (FIG. 2).

### Example (5)

### De-Acm of the Guanylated Peptide Followed by Oxidation to Yield

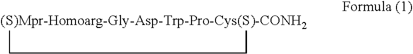

TFA (134.9 ml) and anisole (2.7 ml) are mixed, cooled in ice, added to 658 mg of pre-cooled peptide from example 3 and saturated with nitrogen. This is followed by addition of AgOTf (3.47 g) and stirred for 2 hrs in an ice bath. TFA is removed under high vacuum and silver salt of the peptide was precipitated by addition of diisopropyl ether (˜400 ml). The reaction mixture is filtered through G-4 sintered funnel and precipitate (silver-peptide) is re-suspended in diisopropyl ether (60 ml×3), washed as above and dried over P₂O₅ under vacuum.

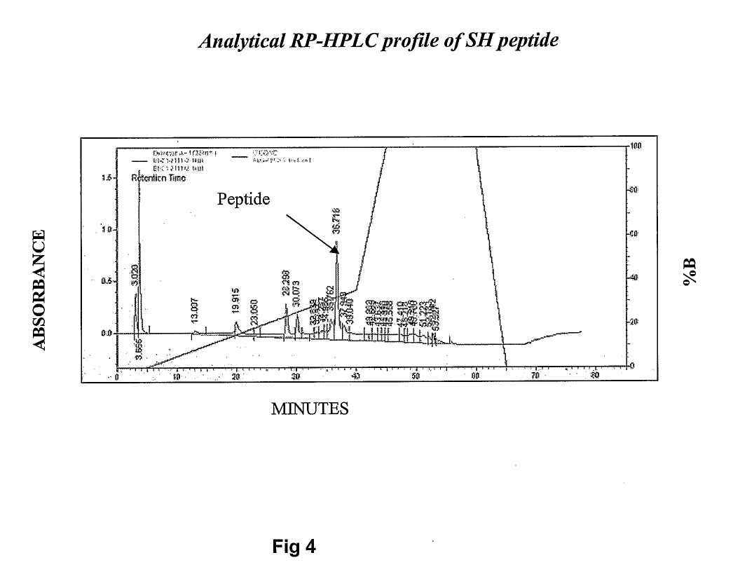

The oxidation silver peptide is carried out by dissolving 10 mg of the silver-peptide salt in 15.6 ml of 50% DMSO/1M HCl in ice-cold condition. The reaction mixture is stirred for 3 hrs at 25° C. The precipitate is filtered through a G-4 sintered funnel or Hyflo bed to remove silver chloride. The filtrate is checked for completion of oxidation (FIG. 4). On completion of the reaction crude peptide of formula (1) is obtained. Percentage purity: 85%

### Example (6)

### Purification of S—S Peptide

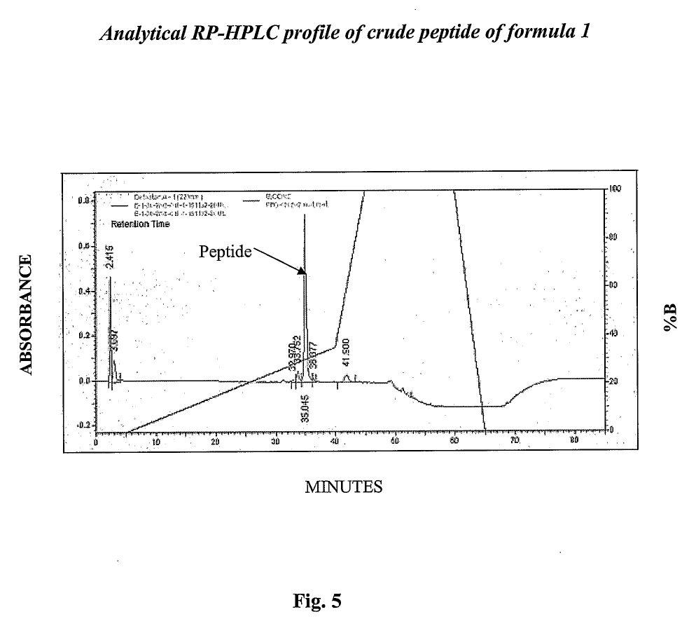

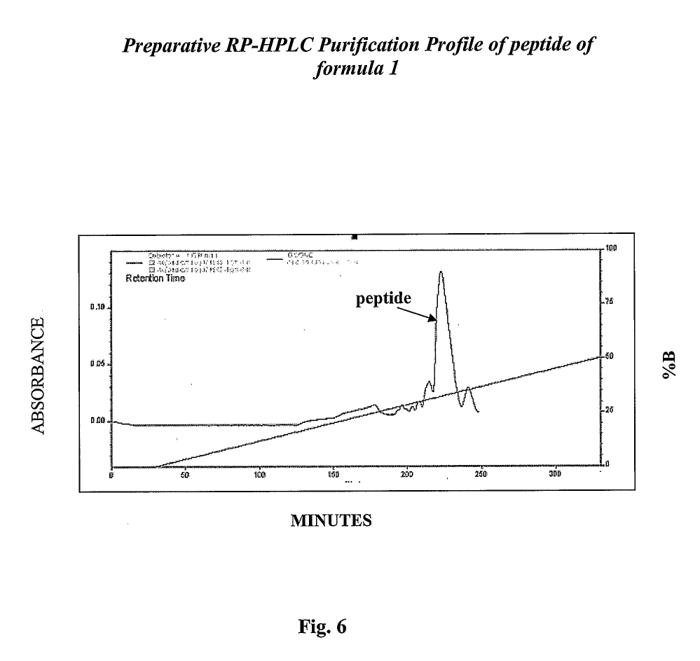

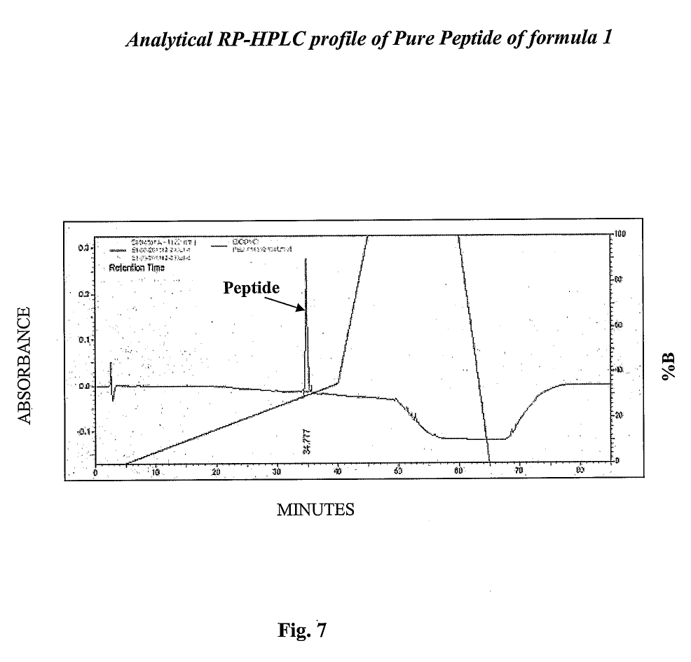

The crude disulfide looped peptide of formula (1) is loaded on to prep C-18 column (50×250 mm, 100 Å). The peptide is purified by using aqueous TFA (0.1%) and methanol in a gradient program (FIG. 5). This is followed by an isocratic run using the above said solvent systems on a Shimadzu preparative HPLC System consisting of a controller, 2 LC8A pumps, UV-Vis detector. The purified peptide amide of formula (1) is analysed by analytical RP-HPLC (FIG. 6). The mass is determined by Mass Spectrophotometer (FIG. 7).

### Example (7)

### Purification of S—S Peptide

The purification was carried out in the same manner as Example 5, except that Acetonitrile was used instead of methanol to obtain peptide amide of formula (1).

### Example (8)

### De-Acm of the Guanylated Peptide Using Mercuric (II) Acetate

Same as in Example (4), except that the Acm group protection of cysteine is removed from the guanylated peptide by treatment with mercury (II) acetate.

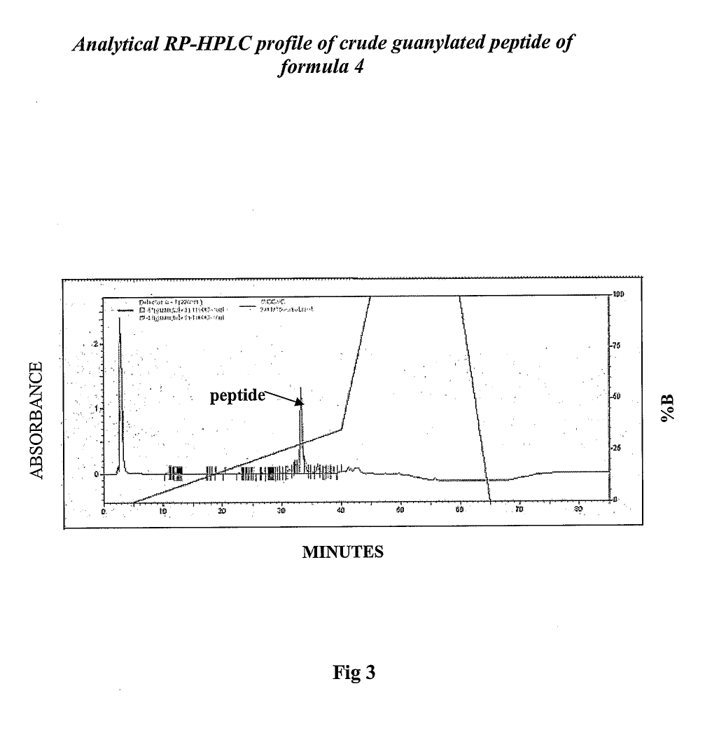

The peptide (13.4 mg) estimated by Lowry's method, of Cys-Acm) is dissolved in 400 μl of acetic acid (10%). Ten times excess of mercury (II) acetate (82.96 mg) is added to it, the reaction mass vortexed and kept at R.T. for 5 hrs. 100 times excess of β-mercaptoethanol (181.37 μl) is added, the solution vortexed and let to stand overnight at room temperature. The reaction mixture is centrifuged for 4 min, and supernatant collected. The precipitate is extracted with 400 μl×3 of 10% acetic acid by centrifugation. The filtrates are pooled and percentage purity determined by RP-HPLC is 55% (FIG. 3).

### Example (9)

### De-Acm of the Guanylated Peptide Using Iodine

Same as in Example (4), except that the Acm group protection of cysteine is removed from the guanylated peptide by treatment with iodine.

The peptide (9.18 mg, estimated by Lowry's method, of Cys-Acm) is dissolved in 17.8 ml of acetic acid (80%) and purged with N₂ for 15 mins. 1 mM solution of I₂ (in 80% acetic acid, ˜4 ml) is added to the peptide solution, over a period of 1 hr, till there is a persistent yellow color. The mixture is stirred for an additional 30-mins followed by neutralisation with 1N Na₂S₂O₃, till the yellow color disappeared, and lyophilized. Estimation of ‘SH’ is done by Ellman Test, which is negative indicating that removal of ACM has not been achieved.

### Example (10)

### Purification of the De-Acm Peptides

The mercury (II) acetate treated and I₂ treated peptide samples were desalted by RP-HPLC, using the hyperprep (250×10 mm, 12μ, C-18 column).

### Example (11)

### Platelet Aggregation Inhibition Assay to Check the Bioactivity of Formula (1)

The bioactivity of peptide of formula (1) is checked using platelet aggregation inhibition assay using 4× Laser Aggregometer (EMA). Freshly venous Blood from consented human donors are drawn and collected in citrated buffer. The platelet rich plasma (PRP) and platelet poor plasma are separated by centrifugation. Platelet count in PRP is adjusted to 2-3×10⁸ platelets/ml. After adjusting the baseline aggregation with PPP, the PRP was treated with 10-20 mM ADP and checked the percent total aggregation. The PRP is then first incubated with varying concentrations of reference standard and synthesized peptide of formula (1). ADP is then added to check the inhibition of aggregation. The reproducibility of bioactivity of synthesized peptide of formula 1 is checked several times and compared with reference standards. Table 1 represents one of many experiments (from 12 experiments). The IC₅₀ dose for synthesized peptide (SP) was less than 140 nM as compared to commercial reference standard. There is more than 50% inhibition of ADP induced platelet aggregation with SP seen in most of the samples and results are comparable with commercial reference standard.

TABLE 1
Inhibition of ADP induced aggregation by synthesized peptide
(SP) of formula 1

| | Percent | | % Inhibition by | |
|---|---|---|---|---|
| Donor | Aggregation by | Concentration | SP of | Reference |
| Number | ADP | (nM) | formula 1 | Standard |
| 1. | 89.4% | 70 | ND | ND |
| | | 140 | 44.6 | 31.20 |
| | | 280 | 61.40 | 52.50 |
| 2. | 92.13 | 70 | 46.66 | 30.31 |
| | | 140 | 51.16 | 45.18 |
| | | 280 | 67.74 | 60.92 |
| 3. | 54.14 | 70 | 57.42 | 27.41 |
| | | 140 | 60.28 | 49.20 |
| | | 280 | ND | ND |
| 4. | 68.11 | 70 | ND | ND |
| | | 140 | 20.52 | 23.06 |
| | | 280 | 42.73 | 40.53 |
| 5. | 66.50 | 70 | 52.63 | 45.68 |
| | | 140 | 87.21 | 57.44 |
| | | 280 | ND | ND |
| 6. | 66.17 | 70 | ND | ND |
| | | 140 | 82.92 | 70.56 |
| | | 280 | ND | ND |

1. A process for the preparation of a peptide N⁶-(aminoiminomethyl)-N²-(3-mercapto-1-oxopropyl-L-lysylglycyl-L-α-aspartyl-L-tryptophyl-L-prolyl-L-cysteinamide, cyclic(1→6)-disulfide of formula (1) on a solid phase, 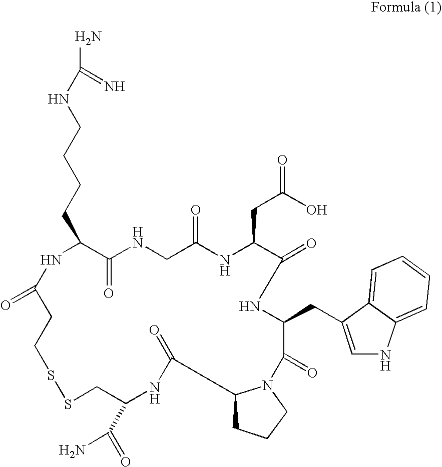
the said process comprising:
a. assembling a peptide chain comprising of six amino acids and a thioalkyl carboxylic acid in a required sequence on a solid support resin, by coupling to directly join one another by peptide bonds to obtain a peptide bound resin of formula (2) as given below:
(Acm)Mpr-Lys(Boc)-Gly-Asp(Obut)-Trp-Pro-Cys(Acm)-Resin Formula (2)
b. capping the free amino groups after each coupling of step (a) with acetic anhydride;
c. cleaving and deprotecting, all groups except Acm group, the peptide of step (a) from the resin to obtain peptide amide of formula (3) as given below:
(Acm)Mpr-Lys-Gly-Asp-Trp-Pro-Cys(Acm)-CONH₂ Formula (3)
d. guanylating the peptide of formula (3) at ε-lysine-NH₂ in an organic solvents followed by precipitating with another solvent to obtain peptide of formula (4) as given below;
(Acm)CysMpr-Homoarg-Gly-Asp-Trp-Pro-Cys(Acm)-CONH₂ Formula (4)
e. treating the peptide of Formula (4) with a heavy metal salt in an salt using an organic solvent to obtain the heavy metal-peptide salt of formula (5); 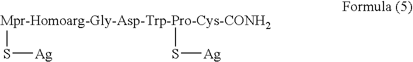
f. Oxidation and desalting of the heavy metal peptide of step (e) with appropriate nucleophilic reagent to obtain the peptide of formula (1); and
g. purifying the peptide of step (f).

2. The process as claimed in claim 1, wherein the reaction of amino and carboxylic equivalent of compounds forms the said peptide bond.

3. The process of claim 1, wherein the C-terminal of the protected first amino acid is bound to a solid phase through a linker to obtain a solid phase bound amino acid.

4. The process of claim 1, wherein the solid support used is amide resin.

5. The process of claim 1, wherein the first protected amino acid is a thiol protected Fmoc Cysteine.

6. The process of claim 1, wherein in the cleavage of the resin with the linker leads to the release of assembled peptide amide.

7. The process of claim 1, wherein the peptide amide compound is a compound joined to each of the terminal functionalities by a peptide bond and wherein each terminal functionalities is an amino or carboxylic acid group or a derivatives thereof.

8. The process of claim 1, wherein the amino acids used are selected from the group consisting of: Cys, Pro, Trp, Asp, Lys, Gly, Arg, Har, Leu and Glu.

9. The process of claim 1, wherein the thioalkyl carboxylic acid used is 2 thiopropionic acid.

10. The process of claim 1 wherein, the protecting group for —NH₂ functional group of an amino acid is Fmoc or Boc.

11. The process of claim 1, wherein the protecting group for the —COOH functional group is O-tBu ester.

12. The process of claim 1, wherein the protecting group for SH-function is Acm group.

13. The process of claim 1, wherein in step (c), the peptide is cleaved from solid support resin using the reagents TFA, TIS, EDT, DCM, Phenol and water.

14. The process of claim 1, wherein in step (d), the organic solvent used for guanylation is selected from the group consisting of DMF, ethanol and methanol.

15. The process of claim 1, wherein in the step (d), the precipitation of the peptide of formula 4 is carried out by using a solvent selected from the group consisting of acetone, acetonitrile, methanol, ethers, pentane, hexane and mixture thereof.

16. The process as claimed in claim 15, wherein the precipitation is performed using acetonitrile.

17. The process of claim 1, wherein in the step (g), the peptide of formula (1) obtained has purity of at least about 99%.

18. The process as claimed in claim 1, wherein the preparation of the peptide of formula (1) by solid phase synthesis is carried out using Fmoc chemistry.

19. The process as claimed in claim 1, wherein the assembly of the amino acids gives a peptide bound resin of formula (2),
(Acm)Mpr-Lys(Boc)-Gly-Asp(Obut)-Trp-Pro-Cys(Acm)-Resin Formula (2)

20. The process as claimed in claim 1, wherein in the step (d), the guanylation of peptide of formula (3) is performed by using the solvent in DMF.

21. A process as claimed in claim 1, wherein the purification of the peptide of Formula (4) is carried out by RP-HPLC.

22. The process as claimed in claim 1, wherein the heavy metal salt used for the treatment of peptide of Formula (4) is silver trifluoromethane sulphonate in TFA.

23. The process as claimed in claim 1, wherein the precipitation of the heavy metal-peptide salt of Formula (5) is carried out using ethereal solvent.

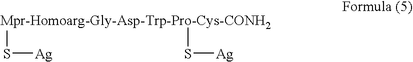

24. The process as claimed in claim 1, wherein in step (f), the heavy metal-peptide salt is treated with HCl and DMSO to simultaneously remove the heavy metal and to oxidize the resulting peptide to yield a crude peptide amide of Formula (1).

25. The process as claimed in claim 1, wherein the crude peptide amide of Formula (1) is purified by RP-HPLC.

26. The process as claimed in claim 1, wherein the purification of crude peptide amide of formula (1) is performed by RP-HPLC using C-4, C-8 or C-18 silica or polymer reverse phase columns using methanol and/or acetonitrile in isolation or combination with aqueous TFA(0-0.5%) as the mobile phase.

27. The peptide of formula (2)
(Acm)Mpr-Lys(Boc)-Gly-Asp(Obut)-Trp-Pro-Cys(Acm)-Resin Formula (2)

28. The peptide of formula (3)
(Acm)Mpr-Lys-Gly-Asp-Trp-Pro-Cys(Acm)-CONH₂ Formula (3)

29. The peptide of formula (4)
(Acm)Mpr-Homoarg-Gly-Asp-Trp-Pro-Cys(Acm)-CONH₂ Formula (4)

30. The peptide salt of the formula (5)

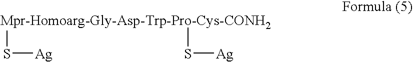

31. A method to improve the yield of a small to medium chain cyclic polypeptide synthesized using solid-phase Fmoc polypeptide synthesis, comprising:
a. providing a solid-phase support,
said solid-phase support bearing bound Fmoc residue,
said bound Fmoc residue substantially free of bound amino acid;
b. synthesizing on said bound Fmoc residue a small to medium chain polypeptide containing at least two sulfur moieties;
c. exposing said sulfur moieties to heavy metal to form sulfur-heavy metal complexes,
d. exposing said sulfur-heavy metal complexes to a nucleophilic reagent, thereby forming a disulfide bond between said sulfur residues, thereby effecting oxidative cyclization of said small to medium chain polypeptide to form a small to medium chain cyclic polypeptide.

32. The method of claim 31, wherein said heavy metal is a heavy metal salt.

33. The method of claim 32, wherein said heavy metal is silver.

34. The method of claim 31, wherein said nucleophilic reagent is a mixture of hydrochloric acid and DMSO.

35. The method of claim 33, wherein said small to medium chain cyclic polypeptide is eptifibatide.

36. In a method for the solid-phase Fmoc polypeptide synthesis on a solid-phase support bearing bound Fmoc residue of a small to medium chain, cyclic polypeptide containing a disulfide bond, the improvement comprising forming said disulfide bond without the use of iodine, whereby the resulting small to medium chain, cyclic polypeptide containing a disulfide bond is substantially free of iodine-related impurities.

37. The method of claim 36, wherein said disulfide bond is formed using a heavy metal.

38. The method of claim 37, wherein said heavy metal is a silver salt.

39. The method of claim 37, wherein said disulfide bond is formed by exposing said heavy metal to a nucleophilic reagent.

40. The method of claim 39, wherein said nucleophilic reagent is DMSO.

41. The method of claim 40, wherein said small to medium chain cyclic polypeptide is eptifibatide.

42. Eptifibatide having a percent inhibition of adenosine diphosphate-induced platelet aggregation, as measured by the platelet aggregation inhibition assay performed on the serum samples of each of six randomly-selected healthy human blood donors, each of said six serum samples adjusted to have a platelet count of 2-3×10⁸ platelets per mL, which percent inhibition for all six said serum samples averages least about 10% greater than the percent inhibition which is shown by reference standard eptifibatide.

43. Eptifibatide having a percent inhibition of adenosine diphosphate-induced platelet aggregation, as measured by the platelet aggregation inhibition assay performed on the serum samples of each of six randomly-selected healthy human blood donors, each of said six serum samples adjusted to have a platelet count of 2-3×10⁸ platelets per mL, which percent inhibition for all six said serum samples, selected from the group consisting of:
(a) a percent inhibition at least about 10% greater than the percent inhibition shown by the reference standard when the concentration of eptifibatide used is 70 nM; or
(b) a percent inhibition at least about 20% greater than the percent inhibition shown by the reference standard when the concentration of eptifibatide used is 140 nM; or
(c) a percent inhibition at least about 50% greater than the percent inhibition shown by the reference standard when the concentration of eptifibatide used is 280 nM.

44. The eptifibatide of claim 43, wherein said eptifibatide displays an average percent inhibition of least about 10% greater than the percent inhibition shown by the reference standard when the concentration of eptifibatide used is 70 nM.

45. The eptifibatide of claim 43, wherein said eptifibatide displays an average percent inhibition of at least about 20% greater than the percent inhibition shown by the reference standard when the concentration of eptifibatide used is 140 nM.

46. The eptifibatide of claim 43, wherein said eptifibatide displays an average percent inhibition of at least about 50% greater than the percent inhibition shown by the reference standard when the concentration of eptifibatide used is 280 nM.

47. The eptifibatide of claim 43, wherein said eptifibatide displays an average percent inhibition selected from two of the group consisting of:
(a) at least about 10% greater than the percent inhibition shown by the reference standard when the concentration of eptifibatide used is 70 nM; and
(b) at least about 20% greater than the percent inhibition shown by the reference standard when the concentration of eptifibatide used is 140 nM; and
(c) at least about 50% greater than the percent inhibition shown by the reference standard when the concentration of eptifibatide used is 280 nM.

48. The eptifibatide of claim 43, wherein said eptifibatide displays an average percent inhibition consisting of:
(a) at least about 10% greater than the percent inhibition shown by reference standard eptifibatide when the concentration of eptifibatide used is 70 nM, and
(b) at least about 20% greater than the percent inhibition shown by reference standard eptifibatide when the concentration of eptifibatide used is 140 nM, and
(c) at least about 50% greater than the percent inhibition shown by reference standard eptifibatide when the concentration of eptifibatide used is 280 nM.
